(19) United States
(12) Patent Application Publication
(10) Pub. No.: US 2025/0114748 A1
(43) Pub. Date: Apr. 10, 2025

(54) MEMBRANE-BASED LIQUID-LIQUID EXTRACTION METHOD

(71) Applicant: KING FAHD UNIVERSITY OF PETROLEUM AND MINERALS, Dhahran (SA)

(72) Inventors: Muhammad SAJID, Dhahran (SA); Mazen Khaled NAZAL, Dhahran (SA); Durga Rao GIJJAPU, Dhahran (SA)

(73) Assignee: KING FAHD UNIVERSITY OF PETROLEUM AND MINERALS, Dhahran (SA)

(21) Appl. No.: 18/482,439

(22) Filed: Oct. 6, 2023

(51) Int. Cl. B01D 61/24; B01D 11/04; B01D 63/04; G01N 1/34; G01N 1/40; G01N 33/487

(52) U.S. Cl. CPC B01D 61/246; B01D 11/0415; B01D 63/043; G01N 1/34; G01N 33/487; B01D 2313/025; B01D 2313/90; B01D 2315/06; G01N 2001/4016

(57) ABSTRACT

A membrane-based inverted liquid-liquid extraction method for extracting an analyte from an unsupported aqueous liquid sample includes sealing the unsupported aqueous liquid sample in a porous membrane bag, immersing the porous membrane bag in an organic solvent, and extracting the analyte from the unsupported aqueous liquid sample to produce an extract within the organic solvent. The unsupported aqueous liquid sample is immiscible with the organic solvent. The porous membrane bag does not contain a solid sorbent.

### STATEMENT REGARDING PRIOR DISCLOSURE BY THE INVENTORS

Aspects of this technology are described in M. Sajid, M. Khaled Nazal, and D. R. Gijjapu, “Membrane-based inverted liquid-liquid extraction of organochlorine pesticides in aqueous samples: evaluation, merits, and demerits” published in Chemical Papers, Volume 77, 3003-3013, which is incorporated herein by reference in its entirety.

### STATEMENT OF ACKNOWLEDGEMENT

This research was supported by the Applied Research Center of Environment and Marine Studies, Research Institute, at King Fahd University of Petroleum and Minerals Dhahran, Saudi Arabia.

### BACKGROUND

### Technical Field

The present disclosure is directed towards a liquid-liquid extraction method, particularly, to a membrane-based inverted liquid-liquid extraction method for extracting the analyte from an unsupported aqueous liquid sample.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Organochlorine pesticides (OCPs) represent a sub-class of organic pollutants that are toxic and known for their persistence in the environment (Zhao L. et al., Journal of Chromatography A 919 (2), 381-388, 2001; Kafilzadeh F., Achievements in the Life Sciences 9 (2), 107-111, 2015). They are toxic and known for their persistence in the environment. OCPs have found extensive use as agrochemicals and in various other products, resulting in their widespread distribution throughout ecosystems. Therefore, it is of significant importance to monitor OCP levels in various matrices using suitable extraction and analytical methodologies.

In spite of remarkable advancements in analytical instrumentation, sample preparation remains a vital step in the development of analytical procedures. Sample preparation is often beneficial when dealing with complex matrices, low analyte concentrations, or analyte-instrument incompatibility. The complex matrix issue is resolved by cleaning up the matrix components (Buszewski, B., & Szultka, M. (2012). Past, Present, and Future of Solid Phase Extraction: A Review. Critical Reviews in Analytical Chemistry, 42(3), 198-213). The low quantities of analytes are concentrated into a volume of extraction phase smaller than the original sample volume by enrichment. The issue of analyte-instrument incompatibility is solved by converting analytes to instrument-compatible derivatives.

Solid-phase extraction (SPE) and liquid-liquid extraction (LLE) are two sample preparation methods that are often employed. SPE separates analytes from samples by passing them through a solid phase, which is generally packed inside the column. Analytes are transported from aqueous to organic or organic to aqueous phase in LLE by mixing the sample with an immiscible extraction phase (Galuszka, A., Migaszewski, Z. M., Konieczka, P., & Namieśnik, J. (2012). Analytical Eco-Scale for assessing the greenness of analytical procedures. TrAC Trends in Analytical Chemistry, 37, 61-72; and Gutiérrez-Serpa, A., González-Martin, R., Sajid, M., & Pino, V. (2021). Greenness of magnetic nanomaterials in miniaturized extraction techniques: A review. Talanta, 225, 122053).

Despite their extensive usage for a wide range of analytes from various matrices, SPE and LLE present substantial challenges owing to their conventional design and handling of complex samples. As a result, more adaptable iterations of SPE and LLE have emerged to tackle specific challenges associated with these techniques. One notable example is dispersive solid-phase extraction (DSPE), which, in contrast to passing the sample through a sorbent-packed column, involves dispersing sorbent material throughout the sample solution (Anastassiades, M., Lehotay, S. J., Štajnbaher, D., & Schenck, F. J. (2003). Fast and Easy Multiresidue Method Employing Acetonitrile Extraction/Partitioning and “Dispersive Solid-Phase Extraction” for the Determination of Pesticide Residues in Produce. Journal of AOAC International, 86(2), 412-431). It fully utilizes the sorbent's adsorption potential while overcoming the backpressure issue that high surface area sorbents in column format might cause. Solid phase microextraction (SPME) has been developed to perform the SPE using minimal quantities of sorbents. SPME device is fabricated by depositing a small amount of sorbent on a silica fiber or metallic wire. Liquid-phase microextraction (LPME) is a miniaturized version of liquid-liquid extraction. It reduces the sample and solvent volumes significantly.

Solutions for analytical extractions are under continuous development. The conventional LLE procedure often requires a large sample volume, and frequently encounters phase separation issues, especially when dealing with complex sample types. Depending on the complexity of the samples, several cleaning cycles may be necessary for some instances. Cumbersome phase separation and multiple cleaning cycles may significantly reduce analyte recovery. In the realm of microextraction, miniaturizing the size and dimensions of LLE is an attractive study area. Single-drop microextraction and hollow-fiber liquid-phase microextraction are only a few of the microextraction technologies that have been developed. Many of the issues connected with large sample volumes have been reduced due to the development of these technologies. However, the practical challenges of working with complex matrices and the availability of a limited number of extraction phases limit their application in routine analysis.

Encapsulating the liquid samples in a porous membrane bag that allows the analytes to pass through but inhibits the complex matrix components is one way to address the complex matrix and unreliable phase separation problem. This concept has been applied to extract analytes from membrane-packed solid samples to a suitable extraction solvent (Sajid M. et al., Microchemical Journal, 144, 2019). It combines cleanup and extraction into a single step. Consequently, it alleviates the needs for numerous pre- and post-extraction steps such as dissolution, filtration, or centrifugation (Kubica P. et al., Molecules, 24 (24), 4618, 2019). Later, this approach was extended to extract the analytes from solid-supported small-volume liquid samples. These samples were packed inside a membrane bag before being put into a solvent. The extraction process may be facilitated by ultrasonication (Robles, F. et al., Molecules, 24 (23), 4376, 2019).

A membrane-assisted liquid-liquid extraction of organochlorine compounds from an aqueous sample in an organic solvent using a low-density polyethylene membrane is known. The organic extract is completely subjected to Gas Chromatography-Electron Capture Detection using large-volume injection (Hauser B. et al., Journal of Separation Science 24 (7), 551-60, 2001).

A micro-solid-phase extraction technique for the determination of organochlorine pesticides in water media, using a gas chromatography-electron capture detector has been disclosed (Salemi A. et al., Microchemical Journal 144, 215-20, 2019).

An ultrasound-assisted solvent extraction for the extraction of organochlorine pesticides from membrane-protected dried fish samples has been described. The dried samples were packed inside a porous membrane bag which was immersed in a solvent and subjected to ultrasonication. After the extraction process, the sample-containing bag was separated from the extract (Muhammad S. et al., Separations 10 (4), 233, 2023).

U.S. Ser. No. 10/191,061B2 discloses a method for extracting an analyte in a sample, wherein the sample and an aqueous solution are microwave-heated and agitated to produce vapor, and the vapor is extracted into an organic solvent contained in a porous membrane bag situated in the vial. The organic solvent containing the vapor extract may then be analyzed for an analyte with gas chromatography-mass spectrometry.

CN108241027A discloses a method to measure chlorobenzene compounds in water samples. The method comprises using a combination of a sample pretreatment technology of membrane-protected micro solid-phase extraction with gas chromatography.

WO1998042410A1 discloses a continuous, recycling, solvent extraction process for the separation and extraction of organochlorines, comprising contacting a perfluorocarbon-insoluble matrix, with a contaminated hydrophobic liquid or solid by means of a recycling liquid-liquid or solid-liquid continuous extraction apparatus.

CN114923746A discloses a sample membrane separation pre-treatment method for tea pesticide residue detection, comprising extracting a tea sample by water to obtain a tea sample extracting solution, firstly using solid phase extraction membrane to absorb, then extracting by extracting solvent to obtain the tea sample extracting solution, then detecting the pesticide residue by chromatographic analysis.

It is of significant importance to monitor levels of pesticides in various matrices using suitable extraction and analysis approaches. Despite recent advances in this field, there is a crucial need to develop a simplified method to extract and analyze pesticides, e.g., organochlorine pesticides, in liquid samples.

In view of the foregoing, one objective of the present disclosure is to provide a membrane-based inverted liquid-liquid extraction method for extracting an analyte from an unsupported aqueous liquid sample. The porous membrane bag containing the unsupported aqueous liquid sample can be separated after extraction, and the step of phase separation is omitted.

### SUMMARY

In an exemplary embodiment, a membrane-based inverted liquid-liquid extraction (MILLE) method for extracting an analyte from an unsupported aqueous liquid sample is described. The method includes sealing the unsupported aqueous liquid sample in a porous membrane bag, immersing the porous membrane bag in an organic solvent, and extracting the analyte from the unsupported aqueous liquid sample to produce an extract within the organic solvent. In some embodiments, the unsupported aqueous liquid sample is immiscible with the organic solvent. In some embodiments, the porous membrane bag does not contain a solid sorbent.

In some embodiments, the analyte is an organochlorine pesticide (OCP).

In some embodiments, the analyte is at least one OCP selected from the group consisting of hexachlorocyclopentadiene (HCCPD), chloroneb, hexachlorobenzene, alpha-benzenehexachloride (alpha-BHC), beta-benzenehexachloride (beta-BHC), gamma-benzenehexachloride (Lindane), delta-benzenehexachloride (delta-BHC), heptachlor, aldrin, chlorothalonil, acetochlor, heptachlorepoxide, dimethyl tetrachloroterephthalate (DCPA), trans-chlordane, trans-nonachlor, cis-chlordane, dichlorodiphenyldichloroethylene (4,4′-DDE), Endosulfan-I, Dieldrin, Endrin, dichlorodiphenyldichloroethane (4,4′-DDD), chlorobenzilate, Endosulfan-II, dichlorodiphenyltrichloroethane (4,4′-DDT), methoxychlor, Endosulfan sulfate, cis-permethrin, and trans-permethrin.

In some embodiments, the analyte is detected and/or quantified in a range of 0.25 to 80 nanograms (ng) per milliliter (mL) unsupported aqueous liquid sample.

In some embodiments, the analyte is present in the unsupported aqueous liquid sample at a concentration of 0.25 to 40 ng/mL.

In some embodiments, the porous membrane bag contains a porous membrane having an average pore size of 0.05 to 1 micrometer (μm) and an average wall thickness of 10 to 500 μm.

In some embodiments, the porous membrane of the porous membrane bag has an average pore size of about 0.2 μm. In some embodiments, the porous membrane of the porous membrane bag has an average wall thickness of about 157 μm.

In some embodiments, the porous membrane bag contains at least one polymer selected from the group consisting of polypropylene, polyethylene, nylon, polyvinylidene fluoride, and polyethersulfone.

In some embodiments, the porous membrane bag consists of polypropylene.

In some embodiments, the organic solvent is at least one selected from the group consisting of acetone, benzene, cyclohexane (CH), n-hexane (Hex), toluene, iso-octane, heptane, dichloromethane (DCM), chloroform (CF), diethyl ether (DEE), and decane.

In some embodiments, the organic solvent is n-hexane.

In some embodiments, the unsupported aqueous liquid sample sealed in the porous membrane bag further contains one or more acids and/or bases selected from the group consisting of nitric acid, sulfuric acid, phosphoric acid, hydrochloric acid, lithium hydroxide, potassium hydroxide, sodium hydroxide, and tetramethyl ammonium hydroxide.

In some embodiments, the unsupported aqueous liquid sample has a pH of 2 to 10.

In some embodiments, the unsupported aqueous liquid sample sealed in the porous membrane bag further comprises one or more inorganic salts selected from the group consisting of sodium chloride, sodium sulfate, sodium citrate, potassium chloride, potassium sulfate, and potassium citrate.

In some embodiments, the one or more inorganic salts are present in the unsupported aqueous liquid sample sealed in the porous membrane bag at a concentration of 0.1 to 1.5 moles per liter (mol/L).

In some embodiments, 0.02 to 0.2 mL of the unsupported aqueous liquid sample is present per square centimeters (cm²) exterior surface area of the porous membrane bag.

In some embodiments, the method includes extracting the analyte from the unsupported aqueous liquid sample for 5 to 180 minutes (min).

In some embodiments, the method has a limit of detection (LOD) in a range of 0.03 to 0.08 ng of the analyte per mL unsupported aqueous liquid sample.

In some embodiments, the method has an analyte recovery of at least 90%.

In some embodiments, the method further includes removing the porous membrane bag from the organic solvent and drying the organic solvent containing the extract to produce a concentrated extract solution, and directly injecting the concentrated extract solution into a mass spectrometer or a chromatography column, wherein no filtering step and/or centrifuging step is used on the organic solvent, extract, or concentrated extract solution.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a flowchart depicting a membrane-based inverted liquid-liquid extraction method for extracting an analyte from an unsupported aqueous liquid sample, according to certain embodiments;

FIG. 1B illustrates a gas chromatography-mass spectrometry (GC-MS)/MS chromatogram depicting the separation of organochlorine pesticide (OCP), according to certain embodiments;

FIG. 2 illustrates a schematic illustration depicting steps involved in membrane-based inverted liquid-liquid extraction (MILLE), according to certain embodiments;

FIG. 3 illustrates a plot depicting effect of extraction solvent on MILLE of OCP, according to certain embodiments;

FIG. 4 illustrates a plot depicting effect of extraction time on MILLE of OCP, according to certain embodiments;

FIG. 5 illustrates a plot depicting effect of sample pH on MILLE of OCP, according to certain embodiments;

FIG. 6 illustrates a plot depicting effect of salt concentration in sample MILLE of OCP, according to certain embodiments; and

FIG. 7 illustrates GC-MS/MS chromatograms of DW sample and spiked DW samples, according to certain embodiments.

### DETAILED DESCRIPTION

In the drawings, like reference numerals designate identical or corresponding parts throughout the several views. Further, as used herein, the words “a,” “an,” and the like generally carry a meaning of “one or more,” unless stated otherwise.

As used herein, the words “about,” “approximately,” or “substantially similar” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), or +/−20% of the stated value (or range of values). Within the description of this disclosure, where a numerical limit or range is stated, the endpoints are included unless stated otherwise. Also, all values and subranges within a numerical limit or range are specifically included as if explicitly written out.

As used herein, the terms “extraction” and “extracting” refer to enhancement of component(s) over other component(s) in one phase. The terms “extraction” and “extracting” includes, but is not limited to, partial extraction i.e., enhancing the relative amount of the component(s) over the other component(s) without completely removing and/or isolating the component(s) relative to the other component(s). The terms “extraction” and “extracting” also include, but are not limited to, complete extraction i.e., completely removing and/or isolating the component(s) relative to the other component(s). The terms “extraction” and “extracting” also includes, but is not limited to, enhancement of the component(s) over the other component(s) by about 10%, about 20%, about 30%, about 40%, about 50%, about 60%, about 70%, about 80%, about 85%, about 90%, about 95%, about 96%, about 97%, about 98%, or about 99%.

As used herein, the term “liquid-liquid extraction” refers to the extraction of a solute dissolved in a first liquid to a second liquid.

As used herein, the term “solute” refers to materials suspended or dissolved in liquid.

As used herein, the term “miscible” refers to a property of liquids to mix in all proportions, forming a homogeneous solution.

As used herein, the term “immiscible” refers to a property of liquids due to which the liquids do not mix in any proportion; they do not form a solution.

As used herein, the term “analyte” refers to any target substance, which is to be removed from a sample.

As used herein, the term “pesticide” refers to pesticides and metabolites and degradation products thereof.

As used herein, the term “pore size” refers to an average diameter of pores of a membrane.

As used herein, the terms “membrane,” or “porous membrane” generally refer to a barrier that allows specific entities (such as molecules and/or ions) to pass through, while retaining passage of others. In the present disclosure, the “membrane” is not a sorbent for the purposes of the present disclosure.

As used herein, the term “organic solvent” refers to solvent that contain carbon. The term “organic solvent” includes, but is not limited to, acetone, benzene, cyclohexane, n-hexane, toluene, iso-octane, heptane, dichloromethane, chloroform, diethyl ether, decane and combinations thereof.

As used herein, the term “unsupported aqueous liquid sample” generally refers to a single phase liquid that may optionally contain one or more dissolved substances and excludes a solid phase material such as a sorbent. In the present disclosure, the one or more dissolved substances include one or more volatile dissolved solids, e.g., preferably one or more organochlorine pesticides (OCPs). Preferably, if present in addition to one or more OCPs, the one or more dissolved substances is a non-volatile material such as a salt. In a preferred embodiment, the one or more dissolved substances may further include one or more non-volatile dissolved substances, such as one or more acids and/or bases, and one or more inorganic salts. The one or more acids and/or bases, and the one or more inorganic salts are water soluble and non-volatile, and do not react with each other. In some other embodiments, the one or more acids and/or bases, and the one or more inorganic salts may react with each other to form one or more complexes, and one or more salts that are water soluble. In some further embodiments, the unsupported aqueous liquid sample is substantially stable, e.g., the one or more dissolved substances remain substantially unchanged during sample preparation, analyte extraction, and analyte characterization.

Aspects of the present disclosure are directed to a membrane-based inverted liquid-liquid extraction (MILLE) method for the extraction of organochlorine pesticides (OCPs) in aqueous samples. Suitable examples of the OCPs include, but are not limited to, one or more of hexachlorocyclopentadiene (HCCPD), chloroneb, hexachlorobenzene, alpha-benzenehexachloride (alpha-BHC), beta-benzenehexachloride (beta-BHC), gamma-benzenehexachloride (Lindane), delta-benzenehexachloride (delta-BHC), heptachlor, aldrin, chlorothalonil, acetochlor, heptachlorepoxide, dimethyl tetrachloroterephthalate (DCPA), trans-chlordane, trans-nonachlor, cis-chlordane, dichlorodiphenyldichloroethylene (4,4′-DDE), Endosulfan-I, Dieldrin, Endrin, dichlorodiphenyldichloroethane (4,4′-DDD), chlorobenzilate, Endosulfan-II, dichlorodiphenyltrichloroethane (4,4′-DDT), methoxychlor, Endosulfan sulfate, cis-permethrin, and trans-permethrin.

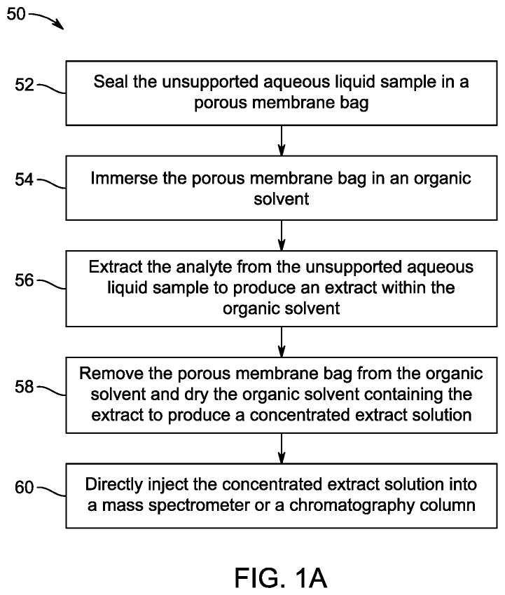

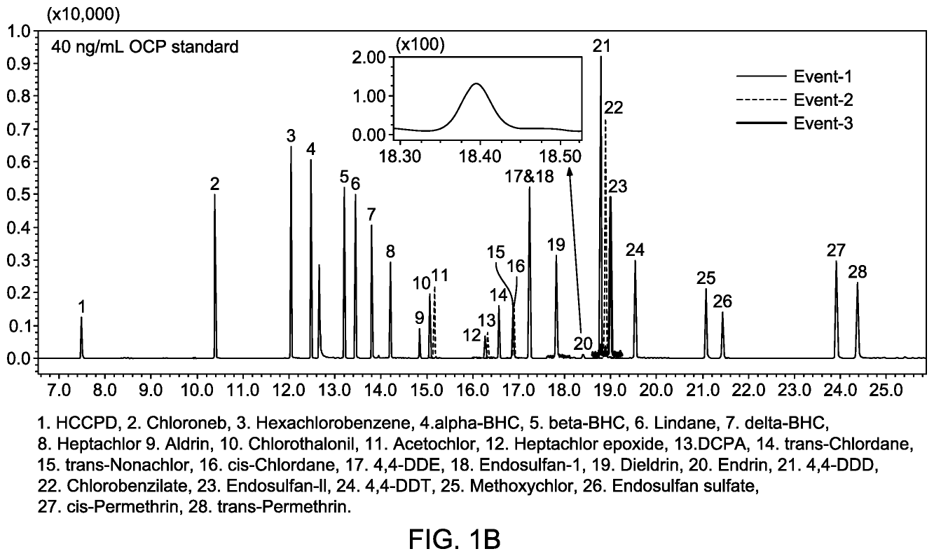

FIG. 1A illustrates a flow chart of method 50 of a membrane-based inverted liquid-liquid extraction (MILLE) method for extracting an analyte from an unsupported aqueous liquid sample. In an embodiment, the analyte is an organochlorine pesticide (OCP). The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes sealing the unsupported aqueous liquid sample in a porous membrane bag. In some embodiments, the unsupported aqueous liquid sample may include the analyte. In an embodiment, the analyte is an organochlorine pesticide (OCP). In some embodiments, the analyte is present in the unsupported aqueous liquid sample at a concentration in a range of 0.05 to 100 ng/mL, preferably 0.1 to 80 ng/mL, preferably 0.2 to 60 ng/mL, or even more preferably 0.25 to 40 ng/mL. Other ranges are also possible.

In addition to one or more OCP's, the unsupported aqueous liquid sample sealed in the porous membrane bag may further include one or more acids, one or more bases, and one or more inorganic salts. In some embodiments, the one or more acids, the one or more bases, and the one or more inorganic salts are water soluble, and completely dissolved in water in the formation of the unsupported aqueous liquid sample. In some further embodiments, the one or more acids, the one or more bases, and the one or more inorganic salts do not react with each other in the formation of the unsupported aqueous liquid sample. In some other embodiments, the one or more acids, the one or more bases, and the one or more inorganic salts may reach with each other to form one or more water soluble complexes and one or more water soluble salts. Suitable examples of acids that may be present in the porous membrane bag may include, nitric acid, sulfuric acid, phosphoric acid, hydrochloric acid, and or combinations thereof. Suitable examples of bases in the porous membrane bag may include, lithium hydroxide, potassium hydroxide, sodium hydroxide, tetramethyl ammonium hydroxide, and/or combinations thereof. In some embodiments, the unsupported aqueous liquid sample sealed in the porous membrane bag further includes one or more inorganic salts. The inorganic salts may be one or more of sodium chloride, sodium sulfate, sodium citrate, potassium chloride, potassium sulfate, and potassium citrate. In some embodiments, the inorganic salts are present in the unsupported aqueous liquid sample sealed in the porous membrane bag at a concentration in a range of 0.01 to 5 moles per liter (mol/L), preferably 0.05 to 3 mol/L, or even more preferably 0.1 to 1.5. Other ranges are also possible. In some embodiments, the unsupported aqueous liquid sample can exist over a wide pH range of 2 to 10, preferably 3 to 9, preferably 4 to 8, or even more preferably 5 to 7. Other ranges are also possible.

In some embodiments, about 0.001 to 1, preferably about 0.005 to 0.8, preferably about 0.01 to 0.6, preferably about 0.015 to 0.4, or even more preferably about 0.02 to 0.2 mL of the unsupported aqueous liquid sample is present per square centimeters (cm²) exterior surface area of the porous membrane bag. Other ranges are also possible. In some embodiments, the porous membrane bag is made up of at least one polymer selected from polypropylene, polyethylene, nylon, polyvinylidene fluoride, and polyethersulfone. In a preferred embodiment, the polymer is polypropylene, though other synthetic membranes with similar thickness and porosity may be used. Polypropylene may be a homopolymer or a copolymer, such as a block copolymer or random copolymer. Conveniently, a commercially available polypropylene may be used. In an alternative embodiment, more than one polymer may comprise the porous membrane. In a more preferred embodiment, the polypropylene has a molecular weight in a range of 20,000 to 1,000,000 g/mol, preferably 50,000 to 700,000 g/mol, preferably 100,000 to 500,000 g/mol, or even more preferably about 300,0000 g/mol. Other ranges are also possible. The porous membrane may be composed of both polypropylene and polyethylene with a polypropylene to polyethylene weight ratio range of 1:10-10:1, preferably 1:7-7:1, preferably 1:5-5:1, or even more preferably 1:2-2:1. Other ranges are also possible.

In some further embodiments, the porous membrane bag includes a porous membrane having an average pore size of about 0.05 to 1 micrometer (μm), preferably 0.07-0.9 μm, preferably 0.09-0.7 μm, preferably 0.1-0.6 μm, preferably 0.12-0.5 μm, preferably 0.0.15-0.3 μm, preferably 0.18-0.2 μm, preferably 0.1-0.2 μm. In some embodiments, the average pore size in the porous membrane will be greater than or equal to 0.05, 0.06, 0.07, 0.08, 0.09 or 0.10 μm and the average thickness will not exceed 100 μm. Other ranges are also possible. In some more preferred embodiments, the porous membrane bag includes a porous membrane having an average wall thickness of about 10 to 500 μm, preferably 20-490 μm, preferably 30-350 μm, preferably 40-340 μm, preferably 50-320 μm, preferably 60-300 μm, preferably 70-280 μm, preferably 90-250 μm, preferably 100-230 μm, preferably 130-200 μm, preferably 140-180 μm, preferably 150-160 μm, preferably 157 μm. Other ranges are also possible.

In one embodiment, the porous membrane forming the porous membrane bag has a uniform thickness of about 100 μm and a pore size of about 1.0 μm. In some embodiments, a polypropylene bag may be replaced by a porous bag made of a different kind of plastic or semipermeable membrane. Preferably the porous membrane has a uniform thickness and pore size that does not vary by more than 0.1, 0.2, 0.5, 1, 2, 3, 4, 5, 6, 7, 8, 9, or 10% over a surface of the porous membrane exposed to the organic solvent. Unlike conventional membranes which do not have uniform thickness or porosity, the use of a porous membrane with uniform properties may provide a more accurate and precise analyte extraction from solid samples.

In one embodiment, the porous membrane itself will be a single layer and the porous membrane bag will not incorporate or contain a solid sorbent or an adsorbent. Examples of solid sorbents include, but are not limited to, silica gel, activated carbon, fly ash, diatomaceous earth, alumina (Al₂O₃), magnesium oxide (MgO), titanium oxide (TiO₂), and polymer sorbents. In a further embodiment, the porous membrane bag does not contain a solid sorbent (the porous membrane bag is not a sorbent for the purposes of the present disclosure). However, in an alternative embodiment, the porous membrane bag may incorporate an adsorbent such as a polymeric matrix containing carbon nanotubes. In this alternative embodiment, it is not necessary for the porous membrane to contain a second layer such as a backing or support layer or a film or applied coating. Such a porous membrane when configured as an enclosure for an unsupported aqueous liquid sample may not contain any loose or packed adsorbent. However, in some alternative embodiments, an adsorbent may be bound to or coated on the porous membrane.

The porous membrane bag may be formed by a single layer of the membrane without additional membranous layers, coatings, films, filtration assemblies, holders, or other external components since a single layer of the porous membrane provides for more rapid and complete perfusion of the organic solvent and extract. Thus, for use, it may be unnecessary to insert the membrane bag into a frame, carrier, filter cartridge assembly, or other mechanical device to affix a filter. However, in some alternative embodiments, two, three, or more layers of porous membrane or may be used to form a membrane bag or the porous membrane bag may be incorporated as part of a larger system comprising other elements such as filters, holders, or other external components.

In one embodiment, the porous membrane bag has a shape that is triangular, tetrahedral, square, cubic, rectangular, parallelepiped, circular, spherical, pouch-like, sachet-like, purse-like, or other shape that prevents release of the aqueous liquid sample and interferences from the porous membrane bag. In some embodiments, two or more porous membranes are sealed together to produce the porous membrane bag, for example, two rectangular or square-shaped porous membranes may have an aqueous liquid sample placed between their surfaces and then sealed to produce a membrane bag. In another embodiment, two circular or oval-shaped membranes may be loaded with the aqueous liquid sample and sealed to product a circular or oval-shaped pod. In other embodiments, only a single membrane will be used to produce a porous membrane bag enclosing the aqueous liquid sample. Typically, the porous membrane bag permanently seals in the aqueous liquid sample so as to prevent its accidental escape into the bulk of the organic solvent.

In some embodiments, the membrane bag comprises pleats or folds that permit the membrane bag to open or expand after contact with the organic solvent, or when holding a sample, thus exposing more of the membrane surface to the organic solvent to be treated. A porous membrane bag may additionally include a seal and a string, thread, or grip to permit it to be dropped into a liquid and then removed. The seal may be a string that secures the contents of the membrane bag at one end by a knot, such as a fiber knot to help shape the bag, or other attachment where the appendage is long enough to permit dipping, swirling, lifting, or other movement of the porous membrane bag in an organic solvent and for removal of the membrane bag from a sample holder after the sonication. A top or distal end of the string, thread or grip, may be attached to a tab which may be colored coded or labelled to permit easy handling and identification of the membrane bag and the aqueous liquid sample. The string, thread, or grip may be about 4, 5, 6, 7, 8, 9, 10, 11, or 12 cm long, or may be longer or shorter depending on the shape of the sample holder. The string, thread or grip may be made of cotton or another natural fiber, a synthetic fiber such as nylon, or a blend of natural and synthetic fibers which may be woven.

In related embodiments, the porous membrane bag may comprise a membrane in the shape of a tube, for example, a hollow fiber membrane, where the ends of the tube are closed in order to contain the aqueous liquid sample. The edges may be closed by an adhesive, a clamp, a tie, or by heat sealing. Alternatively, the membrane may form a balloon shape around the solid sample, with the membrane closed at one side, or with the membrane edges tied at one point. Alternatively, the membrane bag may form a rectangular pillow shape around the aqueous liquid sample. In other embodiments, the edges or perimeter of the membrane may be sealed with an adhesive or folded and mechanically sealed, for example with stitching or stapling. In this embodiment, the four edges may be sealed along each edge, or one edge may be a fold in the membrane with the remaining edges being sealed along each edge. In this pillow shape, the edges may measure 0.4-2 cm, preferably 0.6-1.5 cm, more preferably 0.7-1 cm in length, and the height may be 0.4-2 cm, preferably 0.6-1.5 cm, more preferably 0.7-1 cm. In one embodiment, the length may be about 0.8 cm, and the height may be about 0.8 cm. However, in some embodiments, one or more edges may be less than 0.4 cm or greater than 2 cm.

The porous membrane bag may hold a maximum volume of 0.05-5 cm³, preferably 0.1-1 cm³, more preferably 0.25-0.75 cm³ of an aqueous liquid sample. In one embodiment, 0.02-0.2 mL of the aqueous liquid sample is present per cm² exterior surface area of the porous membrane bag. In other embodiments, preferably 0.04-0.16 mL, more preferably 0.06-0.12 mL, more preferably 0.08-0.12 mL of aqueous liquid sample is present per cm² exterior surface area of the porous membrane bag. Other ranges are also possible. Preferably, of the exterior surface area of the porous membrane bag, at least 80%, preferably at least 90%, more preferably at least 97%, even more preferably about 100% of the is in contact with the organic solvent. Other ranges are also possible.

The membrane bag may enclose both the aqueous liquid sample and a gas. The gas may be present as a bubble or bubbles, and may comprise air, vaporized organic solvent, or some other compound. A gas in the membrane bag may comprise 0-50 vol %, preferably 0-10 vol %, more preferably 0-1 vol % of the total enclosed volume of the membrane bag. Other ranges are also possible.

At step 54, the method 50 includes immersing the porous membrane bag in an organic solvent. Suitable examples of the organic solvent include, acetone, benzene, cyclohexane (CH), n-hexane (Hex), toluene, iso-octane, heptane, dichloromethane (DCM), chloroform (CF), diethyl ether (DEE), and decane. In a preferred embodiment, the solvent is Hex.

At step 56, the method 50 includes extracting the analyte from the unsupported aqueous liquid sample to produce an extract within the organic solvent. The porous membrane bag does not ‘filter’, but rather serves as an artificial interface between the two liquid phases—where one phase includes the unsupported aqueous liquid sample containing the analyte, and the other phase is the organic solvent. The porous membrane bag permits the perfusion of organic solvent and extract/analyte, while confining rest of the unsupported aqueous liquid sample and interferences within the porous membrane bag. In some embodiments, interferences may be solids, mineral complexes, proteins, biomolecules, fiber, humic acid, and/or microorganisms. The unsupported aqueous liquid sample is immiscible with the organic solvent. During the extraction process, the analyte from the unsupported aqueous liquid sample inside the porous membrane bag diffuse through the membrane and into the extraction solvent, where the analyte becomes dissolved.

In some embodiments, the extracting may be performed by shaking, stirring, and/or sonicating the porous membrane bag containing the unsupported aqueous liquid sample in a vial. The extracting may be performed by any other suitable techniques that are known to those skilled in the art. In some embodiments, the extraction process is carried out for 5 to 600 minutes, preferably 10 to 480 minutes, preferably 20 to 360 minutes, preferably 30 to 240 minutes, or even more preferably 40 to 180 minutes, with the extraction time dependent on the organic solvent. Other ranges are also possible. In an embodiment, the extraction process is carried out for 5-60 minutes when the solvent is hexane.

As used herein, the term “shake,” or “shaking” generally refers to a rapidly and repeatedly moving an object or substance back and forth or up and down, often with the intent of mixing, blending, dispersing, or agitating its components. This movement is typically characterized by a series of quick, oscillatory motions. In the present disclosure, the term “shaking” generally refers to mixing the porous membrane bag containing the aqueous liquid sample in a vial containing the organic solvent for the purpose of extracting the analyte within the aqueous liquid sample to the organic solvent. In one embodiment, the contents within the vial may additionally be agitated by shaking, tilting, or vortexing the vial, or by stirring the organic solvent with a stir bar, a stirring rod, or an impeller. This agitating may come before, during, and/or after the sonicating. In one embodiment, the agitating during or after the sonicating may enable faster diffusion of the analyte through the porous membrane bag. In one embodiment, the porous membrane bag and organic solvent may be stirred with a stir bar. The stir bar may be stirred at a rate of 30-1000 rpm, preferably 60-700 rpm, more preferably 100-500 rpm. In another embodiment, the additional agitation may not be necessary as the analyte diffusion through the porous membrane bag is already complete.

In a preferred embodiment, the extracting may be performed by sonicating the vial containing the organic solvent, the porous membrane bag containing the aqueous liquid sample, using a sonicator. In this embodiment, the extraction time may be considered the duration of the sonication. Here, the vial is sonicated for a period of 5-60 min, preferably 10-50 min, more preferably 15-40 min, 20-30 min, 21-29 min, 22-27 min, even more preferably about 25 min. The sonicating may be continuous, pulsed, or modulated in some way. Preferably the sonicating is continuous. The sonicator used may be a probe sonicator inserted into the mixture, or, more preferably, a bath sonicator that can sonicate a plurality of samples without a direct contact between the sonicator and the samples. Without such a direct contact, the chance of cross-contamination is reduced, and cleaning of the sonicator between different samples is not needed. The sonicating frequency is about 20-120 kHz, about 40-100 kHz, or about 60-90 kHz, and may be considered “ultrasonication.” However, in an alternative embodiment, a sonicator may create vibrations at a lower frequency than ultrasonic, for example, sonic vibrations, which may be used for the same purpose. The sonicating power may be 20-100 W, preferably 40-80 W, 45-75 W, more preferably 50-70 W, even more preferably 55-65 W, or about 60 W. Other ranges are also possible.

In one embodiment of the method, the extraction of the analyte occurs at the same time as shaking, and/or sonicating, which means that the entire extraction method occurs in a single operation. For example, an analyte may be extracted from the aqueous liquid sample inside the porous membrane and into the organic solvent passing thorough the porous membrane, without requiring a researcher to perform those extraction steps separately or subsequently. Additionally, in this embodiment, parts of the analyte may be simultaneously partitioned to three locations within the vial: in the aqueous liquid sample within the porous membrane bag, within the organic solvent within the porous membrane bag, and within the organic solvent outside the porous membrane bag but within the vial. In addition, analyte may be present in the headspace of the vial as a vapor. In another embodiment, the analyte may be limited to different locations in the vial throughout the method. For example, at the beginning of the extracting, the analyte may exist only in the aqueous liquid sample and within the membrane bag. With prolonged extracting, e.g., shaking, analyte may exist in all four places as previously described. Likewise, an internal standard may simultaneously exist in three or all four locations within the vial, or may be limited to one or two locations. In one embodiment, an internal standard may be mixed with the aqueous liquid sample. In other embodiments, an internal standard may be added to the organic solvent outside of or within the porous membrane bag.

The amount of analyte or internal standard in the phases of the sample, solution, vapor, and liquid-phase extraction medium may depend on initial concentrations, the amount and rate of heating and agitating, and the partition coefficient of the analyte or internal standard among the different phases. Where two or more analytes are present, their relative concentrations may vary across different phases. Likewise, where one or more internal standards are present, the relative concentrations between an internal standard and an analyte, or between two internal standards, may also vary across different phases.

In some embodiments, the extracting may be performed under heating at a temperature of 40-80° C., preferably 45-70° C., for 1-4 hours, or by placing in a desiccator or vacuum desiccator. In one embodiment, the aqueous liquid sample may be homogenized by milling or by a mortar and pestle, while in other embodiments the homogenization may be by a blade grinder.

In one embodiment, during the method, a temperature of the organic solvent does not exceed 50° C., preferably does not exceed 48° C., more preferably does not exceed 45° C. In one embodiment, the sample may be cooled during the shaking, and/or sonicating, for instance, by controlling the temperature of the water bath in a bath sonicator. In other embodiments, a maximum temperature reached by the organic solvent may be in a range of 30-80° C., preferably 35-75° C., more preferably 40-70° C., or less than 65° C. In a related embodiment, a maximum temperature reached by the organic solvent may be no greater than 70° C. Other ranges are also possible.

At step 58, the method 50 includes removing the porous membrane bag from the organic solvent and drying the organic solvent containing the extract to produce a concentrated extract solution. The porous membrane bag is further removed from the organic solvent, and the organic solvent is dried to produce a concentrated extract. In some embodiments, the drying may be performed with a rotary evaporator, or by letting the vial sit open on a lab bench or in a fume hood. In one embodiment, the concentrated extract solution is located in a bottom portion of the vial. In some embodiments, the drying is carried out at 20 to 100° C., preferably 30 to 80° C., or even more preferably 40 to 70° C., to produce the concentrated extract solution. Other ranges are also possible.

In another embodiment, the concentrated extract solution may be further dried to form a dried extract. The dried extract may then be resuspended in a second volume of a second organic solvent to produce a second concentrated extract solution, for instance, when the second volume of the second organic solvent has a volume is less than the organic solvent used for the extraction. In one embodiment, the second volume may have a volume that is 0.5-60%, preferably 1.0-30%, more preferably 2.0-20%, even more preferably about 10% of the volume of the organic solvent used for the extraction. In some embodiments, a different organic solvent may be used for the concentrated extract solution as compared to the organic solvent used for the extraction. In alternative embodiments, the extract may be diluted to a greater volume (and thus lower concentration of extract) than the original volume of the organic solvent used for the extraction. Preferably, no filtering step and/or centrifuging step is used on the organic solvent, extract, or concentrated extract solution.

At step 60, the method 50 includes directly injecting the concentrated extract solution into a mass spectrometer or a chromatography column, wherein no filtering step and/or centrifuging step is used on the organic solvent, extract, or concentrated extract solution.

In one embodiment, the mass spectrometer or chromatography column may be part of a GCMS. In one embodiment, a portion of the organic solvent may be injected directly into a GCMS without forming a concentrated extract. In one embodiment, a typical commercial GCMS may be used. The gas chromatography may be coupled with a single mass spectrometer (i.e., GC-MS) or with a plurality of mass spectrometers, i.e., tandem mass spectrometry, such as GC-MS-MS. During the mass spectrometry, the analyte may be fragmented by either positive chemical ionization, for example, with methanol as the chemical ionization reagent, or preferably electron ionization (such as GC-EI-MS). In other embodiments, the detection and quantification of the concentration of analytes in the solid sample is via gas chromatography coupled with a flame ionization detector (FID), a thermal energy detector, a nitrogen-phosphorus detector, or a nitrogen chemiluminescence detector. The carrier gas may be nitrogen, helium, and/or hydrogen. Preferably the carrier gas is helium with a purity of greater than 99.9 mol %, preferably greater than 99.99 mol %, more preferably greater than 99.999 mol %. The stationary phase of the gas chromatography column may be comprised of a methyl siloxane (also known as methyl polysiloxane or dimethyl polysiloxane), phenyl polysiloxane, dimethyl arylene siloxane, cyanopropylmethyl polysiloxane, and/or trifluoropropylmethyl polysiloxane with a film thickness of 0.10-7 μm, preferably 0.15-1 μm, more preferably 0.2-0.5 μm. The column length may be 10-120 m, preferably 15-50 m, more preferably 25-40 m, with an inside diameter of 0.08-0.60 mm, preferably 0.15-0.40 mm, more preferably 0.20-0.30 mm.

In some embodiments, a helium gas flow of purity about 99.999% was used as carrier gas with a flow rate of, e.g., preferably about 1.2 mL/min and the total run time was, e.g., preferably about 27 min. The extract of a volume, e.g., preferably about 1.0 μL was injected using split-less mode. In some embodiments, the injector temperature may be maintained at, e.g., preferably about 300° C. In some embodiments, the column oven temperature was ramped from, e.g., preferably about 70° C. after a holding time of, e.g., preferably about 1 min to, e.g., preferably about 200° C. at a rate of, e.g., preferably about 10° C./min. Then another ramping was set from, e.g., preferably 200° C. to 250° C. at a rate of, e.g., preferably about 5° C./min with a holding time of, e.g., preferably 3 min. The temperature of the detector was set at, e.g., preferably about 250° C. Other ranges are also possible.

The parameters of a GCMS instrument and method of operation, including but not limited to flow rate, temperature, temperature gradient, run time, pressure, sample injection, sample volume, ionization method, ionization energy, and scanning range may be adjusted by a person of ordinary skill in the art to account for differences in samples, equipment, and techniques.

The analyte may be detected by monitoring a known elution time and/or m/z (mass to charge ratio) for a positive signal as compared with a blank sample. An internal standard's m/z may depend on its identity. A standard solution containing a mixture of 29 organochlorine Pesticides (OCPs) at 500 mg/L was purchased from RESTEK (EPA method 525.3 OCP). The analyte may further be quantified with a standard addition of an internal standard, such as benz[a]anthracene-D12. For quantitation, known concentrations of an internal standard may be added to the aqueous liquid sample that is divided into aliquots. These aliquots are each extracted and measured by GCMS. Alternatively, aliquots of an analyte extract from a single trial could receive a standard addition. The linear response of the mass spectrometer counts per concentration of internal standard can be extrapolated to quantify an analyte. Additionally, more than one internal standard can be used in order to span a range of molecular masses. Alternatively, standards may be used to calibrate a GCMS prior to analyzing extracted samples.

In an alternative embodiment, gas chromatography may be used for detection and/or quantitation of an analyte without using mass spectrometry. In a related embodiment, the linear trend of the peak areas of the gas chromatogram may be used for quantitation. Generally, a person of ordinary skill in the art may be able to determine the procedure and calculations to quantify and/or detect an analyte based on GCMS data.

In some embodiments, the analyte is detected and/or quantified in a range of 0.05 to 200, preferably 0.1 to 160, preferably 0.15 to 120, preferably 0.2 to 80, or even more preferably 0.25 to 80 nanograms (ng) per milliliter (mL) unsupported aqueous liquid sample. The method of the present disclosure has a limit of detection (LOD) in a range of 0.03 to 0.08 ng of the analyte per mL unsupported aqueous liquid sample and an analyte recovery of at least 80%, preferably at least 90%, or even more preferably at least 99%. Other ranges are also possible.

The factors affecting the extraction process, such as extraction solvent, extraction time, sample pH, and salt addition, were examined. Analytical figures of the method described in the present disclosure were determined under pre-determined extraction conditions. In some preferred embodiments, the method of the present disclosure showed a good linear working range for all the selected OCPs from, e.g., preferably 0.25 to 80 ng/mL with coefficients of determination up to, e.g., preferably 0.9993. In some more preferred embodiments, the LODs ranged from, e.g., preferably 0.03 to 0.08 ng/mL. Other ranges are also possible. Finally, the applicability of the method described herein was tested for real water samples, and the relative recoveries were within an acceptable range, e.g., preferably 80.8-120.4%. The method of the present disclosure may be used for the analysis of OCPs in routine analytical laboratories.

### EXAMPLES

The following examples demonstrate a membrane-based inverted liquid-liquid extraction (MILLE) method for extracting an analyte from an unsupported aqueous liquid sample, as described herein. The examples are provided solely for illustration and are not to be construed as limitations of the present disclosure, as many variations thereof are possible without departing from the spirit and scope of the present disclosure.

### Example 1: Materials

Polypropylene (PP) membrane sheet (Pore size of preferably about 200 nm (0.2 μm) and thickness of preferably about 157 μm) was obtained from Membrana (Wuppertal, Germany). Analytical grade chemicals and organic solvents were purchased and used, i.e., sodium chloride from Sigma Aldrich, Dichloromethane (DCM) from CARLO ERBA-99%, n-Hexane (Hex) from CARLO ERBA-99%, Chloroform (CF) from Fisher Chemicals, Cyclohexane (CH) from BDH Chemicals-99.5% and Diethyl ether (DEE) from Sigma Aldrich-99.8%. Potassium hydroxide (KOH) 2 M and nitric acid (HNO₃) 2 M obtained from Sigma Aldrich were used to adjust the pH of the solutions. A standard solution containing a mixture of 29 organochlorine Pesticides (OCPs) at 500 mg/L was purchased from RESTEK (EPA method 525.3 OCP), and 28 compounds listed in Table 1 were examined in this work. A working standard solution of OCPs with concentrations of 1 mg/L was prepared in acetone.

### Example 2: Gas Chromatography-Tandem Mass Spectrometry (GC-MS/MS) Method for Detection and Quantitation of Organochlorine Pesticide (OCP)

A multi-reaction monitoring (MRM) method was developed for the detection and quantitation of the OCP using gas chromatography coupled with a tandem mass spectrometer (GCMS-TQ8040NX). An Rtx-CL-pesticides capillary column (30 m×id 0.25 mm×ft 0.25 μm) (Restek, USA) was used for the separation of the OCP. High-purity helium (99.999%) gas was used as carrier gas with a flow rate of 1.2 mL/min and a total run time of 27 min. The concentrated extract solution (1.0 μL) was injected using split-less mode. The injector temperature was maintained at 300° C. The column oven temperature was ramped from 70° C. after a holding time of 1 min to 200° C. at a rate of 10° C./min. Then another ramping was set from 200° C. to 250° C. at a rate of 5° C./min with a holding time of 3 min. The temperature of the detector was set at 250° C.

The list of OCPs considered in the concentrated extract solution for the GC-MS/MS analysis and their MRM transitions are given in Table 1.

Referring now to FIG. 1B, which illustrates a GC-MS/MS chromatogram depicting the separation of the OCP. It can be seen from the chromatogram that all the OCPs were well separated to be detected and quantified using the MRM method.

TABLE 1
List of OCPs used in the sample for GC-MS/MS
method development and their MRM transitions.

| OCP | Retention | MRM | Collision | MRM | Collision |
|---|---|---|---|---|---|
| (Molecular formula, | Time (Rₜ) | transitions | energy | transitions | energy |
| molecular weight) | (min) | (m/z) ions (1) | (CE) | (m/z) ions (2) | (CE) |
| HCCPD | 7.65 | 235.00 > 141.00 | 21 | 237.00 > 119.00 | 18 |
| (C₅Cl₆, 272.76) | | | | | |
| Chloroneb | 10.42 | 191.00 > 141.00 | 9 | 191.00 > 113.00 | 12 |
| (C₈H₈Cl₂O₂, 207.05) | | | | | |
| Hexachlorobenzene | 12.25 | 284.00 > 248.70 | 18 | 284.00 > 214.00 | 27 |
| (C₆Cl₆, 284.8) | | | | | |
| alpha-BHC | 12.52 | 181.00 > 145.00 | 12 | 183.00 > 147.00 | 15 |
| (C₆H₆Cl₆, 290.83) | | | | | |
| beta-BHC | 13.32 | 181.00 > 145.00 | 15 | 183.00 > 147.00 | 15 |
| (C₆H₆Cl₆, 290.83) | | | | | |
| Lindane | 13.62 | 181.00 > 145.00 | 12 | 183.00 > 147.00 | 9 |
| (C₆H₆Cl₆, 290.83) | | | | | |
| delta-BHC | 13.92 | 181.00 > 145.00 | 15 | 183.00 > 147.00 | 15 |
| (C₆H₆Cl₆, 290.83) | | | | | |
| Heptachlor | 14.26 | 272.00 > 237.00 | 12 | 100.00 > 65.00 | 12 |
| (C₁₀H₅Cl₇, 373.32) | | | | | |
| Aldrin | 14.91 | 263.00 > 193.00 | 30 | 263.00 > 228.00 | 21 |
| (C₁₂H₈Cl₆, 364.9) | | | | | |
| Chlorothalonil | 15.17 | 266.00 > 168.00 | 18 | 264.00 > 168.00 | 27 |
| (C₈Cl₄N₂, 265.91) | | | | | |
| Acetochlor | 15.25 | 174.00 > 146.00 | 12 | 146.00 > 131.00 | 12 |
| (C₁₄H₂₀ClNO₂, | | | | | |
| 269.76) | | | | | |
| Heptachlorepoxide | 15.91 | 353.00 > 262.00 | 9 | 355.00 > 265.00 | 9 |
| (C₁₀H₅Cl₇O, 373.32) | | | | | |
| DCPA | 16.31 | 355.00 > 265.00 | 9 | 299.00 > 271.00 | 10 |
| (C₁₀H₆Cl₄O₄, | | | | | |
| 331.95) | | | | | |
| trans-Chlordane | 16.55 | 375.00 > 266.00 | 27 | 373.00 > 266.00 | 30 |
| (C₁₀H₆Cl₈, 409.8) | | | | | |
| trans-Nonachlor | 16.79 | 406.80 > 299.90 | 24 | 375.00 > 268.00 | 16 |
| (C₁₀H₅Cl₉, 444.2) | | | | | |
| cis-Chlordane | 16.92 | 372.90 > 265.00 | 27 | 372.90 > 263.90 | 30 |
| (C₁₀H₆Cl₈, 409.8) | | | | | |
| 4,4′-DDE | 17.32 | 248.00 > 176.00 | 30 | 241.00 > 205.90 | 27 |
| (C₁₄H₈Cl₄, 318.02) | | | | | |
| Endosulfan- I | 17.32 | 248.00 > 176.00 | 45 | 246.00 > 176.00 | 30 |
| (C₉H₆Cl₆O₃S, | | | | | |
| 406.93) | | | | | |
| Dieldrin | 17.92 | 79.00 > 51.00 | 15 | 79.00 > 77.00 | 21 |
| (C₁₂H₈Cl₆O, 380.91) | | | | | |
| Endrin | 18.45 | 263.00 > 193.00 | 30 | 245.00 > 126.00 | 21 |
| (C₁₂H₈Cl₆O, 380.91) | | | | | |
| 4,4′-DDD | 18.88 | 235.00 > 165.10 | 24 | 237.00 > 165.00 | 24 |
| (C₁₄H₁₀Cl₄, 320.04) | | | | | |
| Chlorobenzilate | 18.99 | 251.00 > 138.90 | 15 | 139.00 > 111.00 | 12 |
| (C₁₆H₁₄Cl₂O₃, 325.2) | | | | | |
| Endosulfan- II | 19.15 | 160.00 > 125.00 | 9 | 159.00 > 123.00 | 15 |
| (C₉H₆Cl₆O₃S, | | | | | |
| 406.93) | | | | | |
| 4,4′-DDT | 19.65 | 237.00 > 165.00 | 12 | 235.00 > 165.00 | 27 |
| (C₁₄H₉Cl₅, 354.48) | | | | | |
| Methoxychlor | 21.18 | 227.00 > 141.00 | 27 | 227.00 > 169.00 | 27 |
| (C₁₆H₁₅Cl₃O₂, | | | | | |
| 345.65) | | | | | |
| Endosulfan sulfate | 21.52 | 272.00 > 237.00 | 18 | 274.00 > 237.00 | 12 |
| (C₉H₆Cl₆O₄S, 422.9) | | | | | |
| cis-Permethrin | 24.12 | 183.00 > 153.00 | 15 | 183.00 > 168.00 | 9 |
| (C₂₁H₂₀Cl₂O₃ | | | | | |
| 391.28 | | | | | |
| trans-Permethrin | 24.53 | 183.00 > 153.00 | 15 | 183.00 > 168.00 | 9 |
| (C₂₁H₂₀Cl₂O₃, | | | | | |
| 391.28) | | | | | |

### Example 3: Preparation and Cleaning of Porous Membrane Bag

The membrane bag, with dimensions of 4 cm×3 cm, was prepared by folding into half and heat-sealing open ends of a polypropylene membrane sheet (pore size: about 200 nm; thickness: about 157 μm) (Membrane, Wuppertal, Germany). One side of the membrane bag was kept open to fill the sample solution in it. These membrane bags were then conditioned and cleaned by soaking them in methanol and ultrasonicating for 10 minutes using an ultrasound water bath (BRANSON 8800). The cleaned and dried porous membrane bags were used for all experiments.

### Example 4: Determining the Effect of Extraction Solvent on MILLE Performance

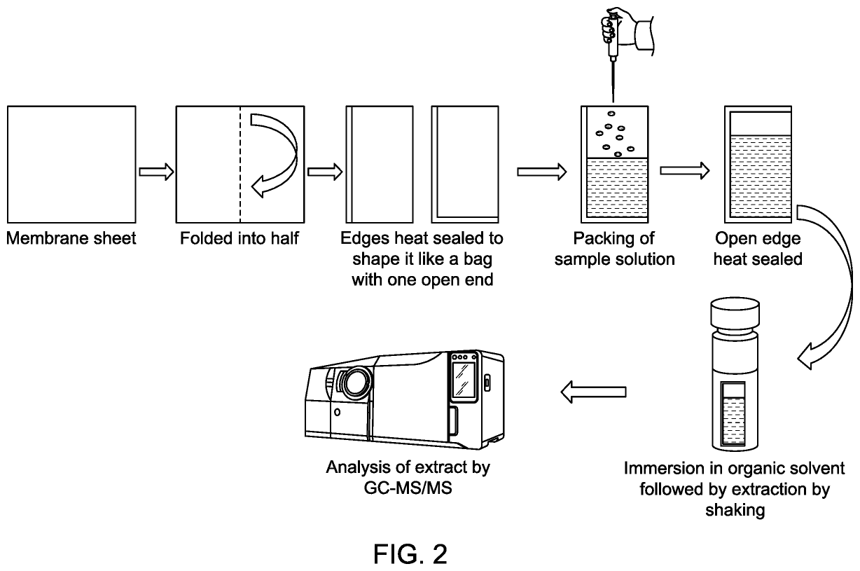

1 mL of deionized water (pH=5.9), including a mixture of OCPs, namely HCCPD, chloroneb, hexachlorobenzene, alpha-BHC, beta-BHC, lindane, delta-BHC, heptachlor, aldrin, chlorothalonil, acetochlor, heptachlorepoxide, DCPA, trans-chlordane, trans-nonachlor, cis-Chlordane, 4,4′-DDE, endosulfan-I, dieldrin, endrin, 4,4′-DDD, chlorobenzilate, endosulfan-II, 4,4′-DDT, methoxychlor, endosulfan sulfate, cis-permethrin, trans-permethrin, at a concentration of 2 ng/mL was packed inside the porous membrane bag from the open end, and this end was then heat-sealed. The sample-packed porous membrane bags were immersed in 2 mL of various organic solvents (DCM, Hex, CF, CH, and DEE) in 40 mL screw-capped glass vials and subjected to the MILLE to obtain the concentrated extract solutions. The concentrated extract solutions were analyzed using the MRM method. FIG. 2 illustrates a scheme depicting the steps involved in the MILLE.

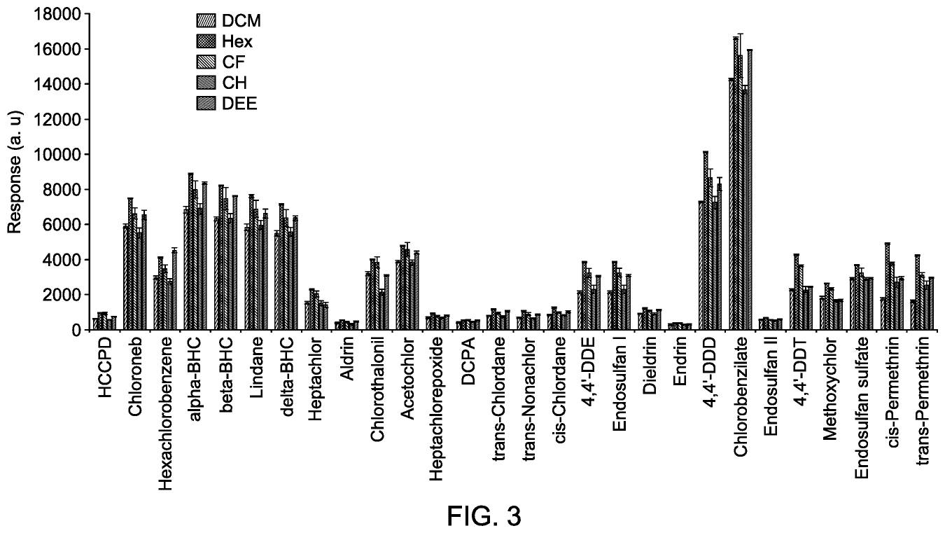

FIG. 3 illustrates a plot depicting the effect of extraction solvent on the MILLE of OCP. Relatively better extraction performance was observed for all the OCPs in the case of Hex. The investigated solvents can be arranged in the following order based on the polarity index: Hex<CH<DEE<DCM<CF. Though extraction results do not appear exactly in the order of the polarity index, the reason for the highest extraction in Hex can be related to its non-polar nature compared to other extraction solvents.

### Example 5: Determining the Effect of Extraction Time on MILLE Performance

1 mL of deionized water (pH=5.9) including a mixture of OCP (HCCPD, chloroneb, hexachlorobenzene, alpha-BHC, beta-BHC, lindane, delta-BHC, heptachlor, aldrin, chlorothalonil, acetochlor, heptachlor epoxide, DCPA, trans-chlordane, trans-nonachlor, cis-Chlordane, 4,4′-DDE, endosulfan-I, dieldrin, endrin, 4,4′-DDD, chlorobenzilate, endosulfan-II, 4,4′-DDT, methoxychlor, endosulfan sulfate, cis-permethrin, trans-permethrin) at a concentration of 2 ng/mL was packed inside the porous membrane bag from the open end, and this end was then heat sealed. The sample-packed porous membrane bags were immersed in 2 mL of the organic solvent in 40 mL screw-capped glass vials. The immersed sample-packed porous membrane bags were subjected to the MILLE for various time durations, i.e., 5 min, 10 min, 15 min, 30 min, 60 min, 120 min, and 180 min, to obtain the concentrated extract solutions. The concentrated extract solutions were analyzed using the MRM method.

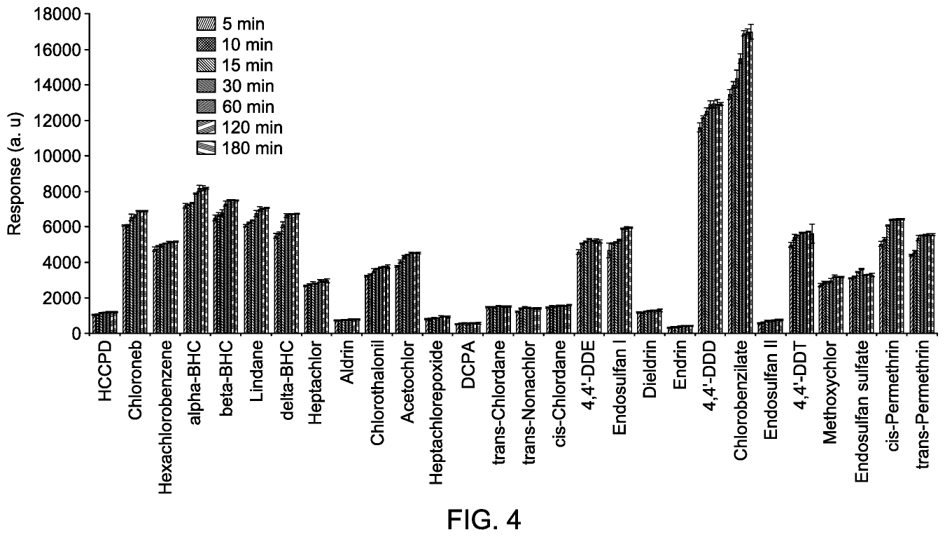

FIG. 4 illustrates the effect of extraction time on the MILLE of OCP. An increase in extraction performance was observed from 5 min to 60 min. However, after this time, no significant increase was recorded in the extraction of OCP, which indicates that an equilibrium state was reached within 60 min.

### Example 6: Effect of Sample pH on MILLE Performance

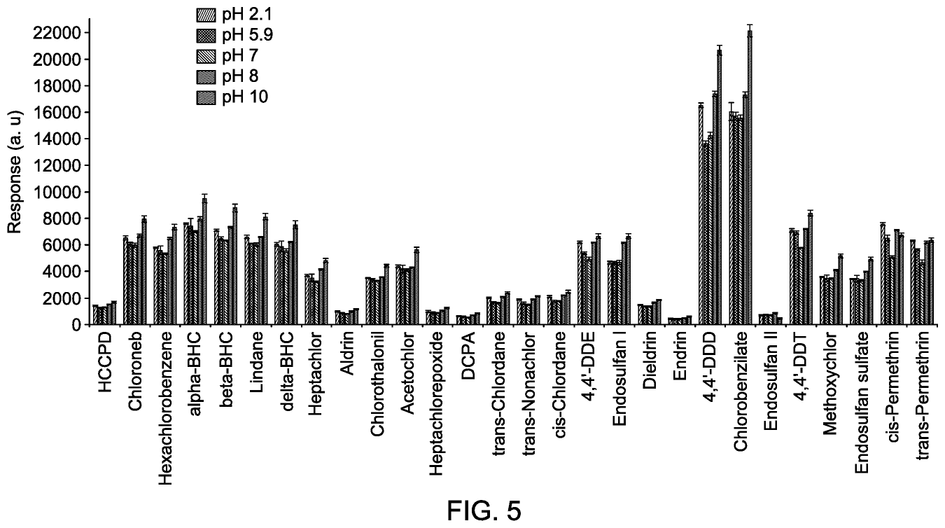

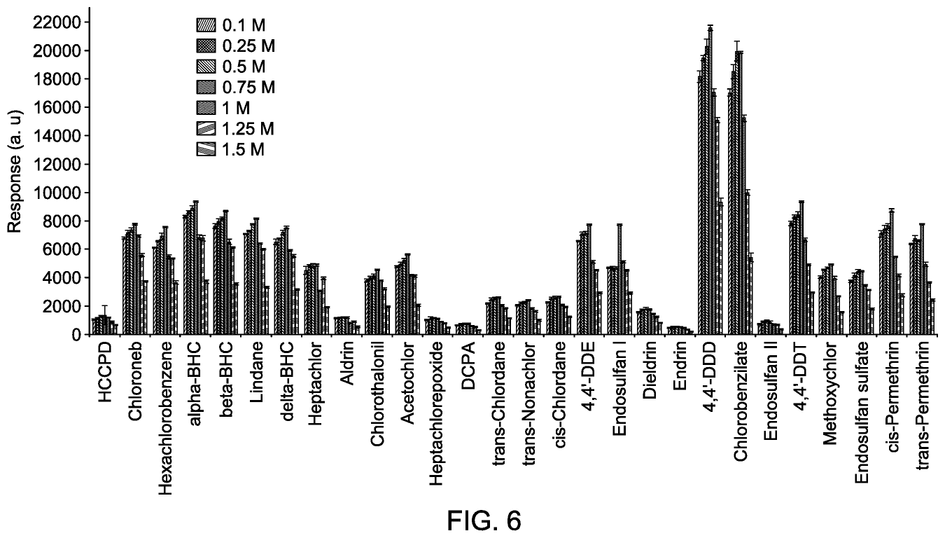

FIG. 5 illustrates a plot depicting the effect of sample pH on the MILLE of OCP. For this purpose, 1 mL of deionized water comprising mixture of OCP (HCCPD, chloroneb, hexachlorobenzene, alpha-BHC, beta-BHC, lindane, delta-BHC, heptachlor, aldrin, chlorothalonil, acetochlor, heptachlorepoxide, DCPA, trans-chlordane, trans-nonachlor, cis-Chlordane, 4,4′-DDE, endosulfan-I, dieldrin, endrin, 4,4′-DDD, chlorobenzilate, endosulfan-II, 4,4′-DDT, methoxychlor, endosulfan sulfate, cis-permethrin, trans-permethrin) at a concentration of 2 ng/mL was used as sample solution. Various batches of the sample solution were adjusted to pH ranging from 2 to 10 (2.1, 5.9, 7, 8, and 10) using 2 molar potassium hydroxide and 2 molar nitric acid solutions. The sample solutions were packed inside the porous membrane bag from the open end, and this end was then heat-sealed. The sample-packed porous membrane bags were immersed in 2 mL of the organic solvent in a 40 mL screw-capped glass vial and subjected to the MILLE to obtain the concentrated extract solutions. The concentrated extract solutions were analyzed using the MRM method. As is evident from the plot, the best extraction performance was observed at sample pH 10.

### Example 7: Effect of Salt Concentration on MILLE Performance

1 mL of deionized water (pH=5.9) including a mixture of OCP (HCCPD, chloroneb, hexachlorobenzene, alpha-BHC, beta-BHC, lindane, delta-BHC, heptachlor, aldrin, chlorothalonil, acetochlor, heptachlorepoxide, DCPA, trans-chlordane, trans-nonachlor, cis-Chlordane, 4,4′-DDE, endosulfan-I, dieldrin, endrin, 4,4′-DDD, chlorobenzilate, endosulfan-II, 4,4′-DDT, methoxychlor, endosulfan sulfate, cis-permethrin, trans-permethrin) at a concentration of 2 ng/mL was used as sample solution. Salt (sodium chloride) was added in various batches of sample at concentrations of 0.1, 0.25, 0.5, 0.75, 1.0, and 1.25 mol/L. The sample solutions were packed inside the porous membrane bag from the open end, and this end was then heat-sealed. The sample-packed porous membrane bags were immersed in 2 mL of the organic solvent in the 40 mL screw-capped glass vial and subjected to the MILLE to obtain the concentrated extract solutions. The concentrated extract solutions were analyzed using the MRM method.

The extraction performance of OCP increased from 0.1 to 0.75 mol/L, and then a gradual decrease was observed. The decrease in extraction may be attributed to the increased viscosity of the sample solution.

### Example 8: Method Validation and Analytical Figures of Merit of MILLE-GC-MS/MS Method: Linearity

Linearity of the MILLE-GC-MS/MS method was tested in concentration ranges of 0.25 to 80 ng/mL (0.25, 0.5, 5, 10, 20, 40, and 80 ng/mL). Calibration curves were developed by extracting a series of OCP standards (HCCPD, chloroneb, hexachlorobenzene, alpha-BHC, beta-BHC, lindane, delta-BHC, heptachlor, aldrin, chlorothalonil, acetochlor, heptachlorepoxide, DCPA, trans-chlordane, trans-nonachlor, cis-Chlordane, 4,4′-DDE, endosulfan-I, dieldrin, endrin, 4,4′-DDD, chlorobenzilate, endosulfan-II, 4,4′-DDT, methoxychlor, endosulfan sulfate, cis-permethrin, trans-permethrin) prepared in water. The samples were subjected to the MILLE under optimum conditions to obtain the concentrated extract solutions. The concentrated extract solutions were analyzed using the MRM method.

The result of the linearity experiment is given in Table 2. The MILLE-GC-MS/MS method exhibited a linear change in response from 0.25 to 80 ng/mL with coefficients of determination (R²) ranging between 0.9729 and 0.9993.

### Example 9: Method Validation and Analytical Figures of Merit of MILLE-GC-MS/MS Method: Sensitivity

The sensitivity of the MILLE-GC-MS/MS method was expressed in terms of its limit of detection (LOD) and limit of quantification (LOQ). A series of OCP standards (HCCPD, chloroneb, hexachlorobenzene, alpha-BHC, beta-BHC, lindane, delta-BHC, heptachlor, aldrin, chlorothalonil, acetochlor, heptachlorepoxide, DCPA, trans-chlordane, trans-nonachlor, cis-Chlordane, 4,4′-DDE, endosulfan-I, dieldrin, endrin, 4,4′-DDD, chlorobenzilate, endosulfan-II, 4,4′-DDT, methoxychlor, endosulfan sulfate, cis-permethrin, trans-permethrin) were prepared in water. The samples were subjected to the MILLE under pre-determined conditions to obtain the concentrated extract solutions. Seven replicates of each sample were spiked at 0.25 ng/mL. The spiked samples were subjected to the MILLE under the pre-determined conditions to obtain the concentrated extract solutions for the determination of LOD and LOQ. LOD and LOQ were calculated using equations (1) and (2), respectively [Ripp J, Analytical detection limit guidance & laboratory guide for determining method detection limits, 1996, which is incorporated herein by reference in its entirety].

$$L O D = ( t - value ) \times {S}_{c} \qquad ( 1 )$$

$$L O Q = 10 \times {S}_{c} \qquad ( 2 )$$

where S_{c} is the standard deviation in the calculated concentration of 5 replicates for samples spiked at 0.25 ng/mL.

The result of sensitivity experiment is given in Table 2. LOD was found to be ranging from 0.03 to 0.08 ng/mL, while LOQ was ranging from 0.09 to 0.26 ng/mL.

### Example 10: Method Validation and Analytical Figures of Merit of MILLE-GC-MS/MS Method: Precision

MILLE-GC-MS/MS method's precision was assessed by calculating intra-day and inter-day precision. Aqueous solution comprising mixture of OCP (HCCPD, chloroneb, hexachlorobenzene, alpha-BHC, beta-BHC, lindane, delta-BHC, heptachlor, aldrin, chlorothalonil, acetochlor, heptachlorepoxide, DCPA, trans-chlordane, trans-nonachlor, cis-Chlordane, 4,4′-DDE, endosulfan-I, dieldrin, endrin, 4,4′-DDD, chlorobenzilate, endosulfan-II, 4,4′-DDT, methoxychlor, endosulfan sulfate, cis-permethrin, trans-permethrin) at a concentration of 2 ng/mL were extracted under optimum conditions to calculate intra-day (n=5) and inter-day (n=5) precision in terms of percentage relative standard deviation (% RSD) of the signal response. The result of the precision experiment is given in Table 2. % RSD for OCP was found to be below 15%.

TABLE 2
Results of method validation and analytical figures of merit of
MILLE-GC-MS/MS method: Linearity, Sensitivity, and Precision

| | | | | | Intra- | Inter- |
|---|---|---|---|---|---|---|
| | | | | | day | day |
| | Linear | | | | pre- | pre- |
| | range | | LOD | LOQ | cision | cision |
| OCP | (ng/ml) | R² | (ng/mL) | (ng/mL) | (%) | (%) |
| HCCPD | 0.25-80 | 0.9759 | 0.05 | 0.16 | 1.9 | 7.9 |
| Chloroneb | 0.25-80 | 0.9893 | 0.04 | 0.14 | 2.0 | 5.2 |
| Hexachlorobenzene | 0.25-80 | 0.9917 | 0.07 | 0.22 | 5.3 | 12.0 |
| alpha-BHC | 0.25-80 | 0.9888 | 0.05 | 0.17 | 7.6 | 4.2 |
| beta-BHC | 0.25-80 | 0.9947 | 0.04 | 0.13 | 1.5 | 7.1 |
| Lindane | 0.25-80 | 0.9979 | 0.05 | 0.16 | 0.3 | 7.2 |
| delta-BHC | 0.25-80 | 0.9975 | 0.04 | 0.14 | 6.9 | 4.3 |
| Heptachlor | 0.25-80 | 0.9953 | 0.04 | 0.14 | 8.1 | 14.4 |
| Aldrin | 0.25-80 | 0.9971 | 0.05 | 0.16 | 4.9 | 5.7 |
| Chlorothalonil | 0.25-80 | 0.9953 | 0.07 | 0.22 | 3.5 | 5.3 |
| Acetochlor | 0.25-80 | 0.9983 | 0.03 | 0.09 | 7.2 | 8.7 |
| Heptachlorepoxide | 0.25-80 | 0.9945 | 0.05 | 0.18 | 8.1 | 10.3 |
| DCPA | 0.25-80 | 0.9852 | 0.07 | 0.24 | 7.5 | 10.5 |
| trans-Chlordane | 0.25-80 | 0.9907 | 0.06 | 0.2 | 2.0 | 8.6 |
| trans-Nonachlor | 0.25-80 | 0.9993 | 0.05 | 0.16 | 6.0 | 9.1 |
| cis-Chlordane | 0.25-80 | 0.9967 | 0.05 | 0.17 | 4.1 | 6.2 |
| 4,4′-DDE | 0.25-80 | 0.9965 | 0.06 | 0.2 | 1.5 | 8.7 |
| Endosulfan- I | 0.25-80 | 0.9957 | 0.05 | 0.15 | 1.8 | 12.7 |
| Dieldrin | 0.25-80 | 0.9728 | 0.08 | 0.26 | 3.6 | 14.7 |
| Endrin | 0.25-80 | 0.9980 | 0.04 | 0.15 | 5.0 | 13.2 |
| 4,4′-DDD | 0.25-80 | 0.9976 | 0.08 | 0.25 | 1.5 | 8.0 |
| Chlorobenzilate | 0.25-80 | 0.9954 | 0.04 | 0.14 | 2.0 | 12.1 |
| Endosulfan- II | 0.25-80 | 0.9958 | 0.04 | 0.12 | 4.9 | 11.5 |
| 4,4′-DDT | 0.25-80 | 0.9920 | 0.05 | 0.15 | 1.7 | 10.9 |
| Methoxychlor | 0.25-80 | 0.9921 | 0.04 | 0.13 | 7.4 | 11.7 |
| Endosulfan sulfate | 0.25-80 | 0.9984 | 0.05 | 0.18 | 6.9 | 11.5 |
| cis-Permethrin | 0.25-80 | 0.9954 | 0.08 | 0.25 | 3.2 | 11.7 |
| trans-Permethrin | 0.25-80 | 0.9983 | 0.04 | 0.13 | 1.1 | 10.7 |

### Example 11: Method Validation and Analytical Figures of Merit of MILLE-GC-MS/MS Method: Accuracy

To check the accuracy of the MILLE-GC-MS/MS method, relative recoveries of analytes were determined by spiking OCP-free water samples (Synthetic wastewater (SWW), Tap water (TW), and Drinking water (DW)) at different concentration levels (0.25, 5, and 40 ng/mL). The relative recoveries were determined using equation (3)

$$Relative Recovery \% = \frac{Concentration after spiking - Concentration without spiking}{Spiked concentration} \times 100 \qquad ( 3 )$$

The result of the accuracy experiment is given in Table 3. The relative recoveries were in the range of 80.8-120.4%, which confirms that the MILLE-GC-MS/MS method can be applied to SWW, TW, and DW samples.

TABLE 3
Result of relative recoveries of OCP from SWW, TW, and DW.

| | SWW | | | TW | | | DW | | |
|---|---|---|---|---|---|---|---|---|---|
| | Spiking level (ng/mL) | | | | | | | | |
| | 0.25 | 5 | 40 | 0.25 | 5 | 40 | 0.25 | 5 | 40 |
| OCP | Relative Recoveries (%) | | | | | | | | |
| HCCPD | 95.1 | 82.6 | 107.5 | 112.2 | 110.1 | 112.3 | 97.0 | 99.2 | 108.6 |
| Chloroneb | 109.1 | 101.1 | 106.2 | 91.7 | 104.6 | 99.8 | 89.0 | 108.4 | 111.1 |
| Hexachlorobenzene | 115.8 | 105.6 | 95.4 | 90.2 | 118.2 | 95.7 | 81.7 | 112.1 | 103.8 |
| alpha-BHC | 106.1 | 99.9 | 103.3 | 112.3 | 114.8 | 97.4 | 99.9 | 119.8 | 111.0 |
| beta-BHC | 108.2 | 105.1 | 103.5 | 103.1 | 119.4 | 96.8 | 100.7 | 110.2 | 105.2 |
| Lindane | 100.3 | 97.6 | 103.1 | 96.9 | 99.6 | 99.3 | 108.8 | 108.5 | 114.9 |
| delta-BHC | 109.1 | 112.1 | 103.2 | 101.4 | 112.5 | 103.0 | 113.0 | 111.6 | 103.0 |
| Heptachlor | 118.3 | 109.6 | 100.8 | 119.6 | 117.4 | 105.4 | 107.7 | 119.7 | 108.1 |
| Aldrin | 100.5 | 100.8 | 101.0 | 109.1 | 116.9 | 112.2 | 93.7 | 119.0 | 113.9 |
| Chlorothalonil | 109.6 | 113.9 | 101.6 | 109.1 | 112.6 | 99.6 | 105.9 | 104.8 | 101.6 |
| Acetochlor | 98.0 | 104.5 | 101.2 | 100.4 | 109.4 | 104.5 | 106.6 | 108.4 | 105.9 |
| Heptachlorepoxide | 101.2 | 97.6 | 104.7 | 114.4 | 109.5 | 94.8 | 107.4 | 111.1 | 113.8 |
| DCPA | 98.3 | 94.3 | 102.4 | 110.0 | 116.4 | 102.7 | 109.4 | 103.6 | 120.0 |
| trans-Chlordane | 114.3 | 107.3 | 100.2 | 99.4 | 110.4 | 101.0 | 94.9 | 105.8 | 110.0 |
| trans-Nonachlor | 110.3 | 84.6 | 104.5 | 89.6 | 107.0 | 111.2 | 104.0 | 119.6 | 113.8 |
| cis-Chlordane | 105.1 | 80.8 | 96.1 | 89.3 | 118.4 | 109.0 | 112.7 | 116.2 | 112.2 |
| 4,4′-DDE | 100.7 | 100.1 | 99.6 | 116.7 | 108.4 | 108.5 | 120.4 | 117.8 | 103.9 |
| Endosulfan I | 103.3 | 102.6 | 101.8 | 116.0 | 118.9 | 102.1 | 119.1 | 116.4 | 115.1 |
| Dieldrin | 115.9 | 99.0 | 103.7 | 109.7 | 113.8 | 105.6 | 89.5 | 113.3 | 113.8 |
| Endrin | 85.5 | 95.9 | 104.1 | 103.0 | 97.7 | 93.7 | 101.8 | 108.6 | 101.1 |
| 4,4′-DDD | 120.4 | 110.3 | 101.5 | 94.5 | 111.2 | 98.9 | 108.5 | 111.3 | 113.6 |
| Chlorobenzilate | 109.4 | 99.6 | 102.5 | 111.1 | 99.6 | 95.6 | 116.2 | 117.7 | 98.1 |
| Endosulfan II | 95.2 | 91.9 | 101.5 | 109.0 | 113.8 | 106.5 | 102.8 | 116.3 | 106.5 |
| 4,4′-DDT | 94.2 | 96.1 | 107.3 | 116.2 | 114.5 | 105.5 | 112.0 | 113.0 | 111.7 |
| Methoxychlor | 118.2 | 99.1 | 103.8 | 117.5 | 118.4 | 100.9 | 107.3 | 110.6 | 109.6 |
| Endosulfan sulfate | 117.5 | 90.2 | 100.2 | 114.9 | 118.0 | 95.5 | 103.1 | 117.8 | 105.5 |
| cis-Permethrin | 89.9 | 86.2 | 105.5 | 117.0 | 113.9 | 109.1 | 108.1 | 115.7 | 119.1 |
| trans-Permethrin | 96.5 | 88.2 | 103.7 | 85.7 | 115.9 | 107.3 | 106.7 | 118.6 | 113.4 |

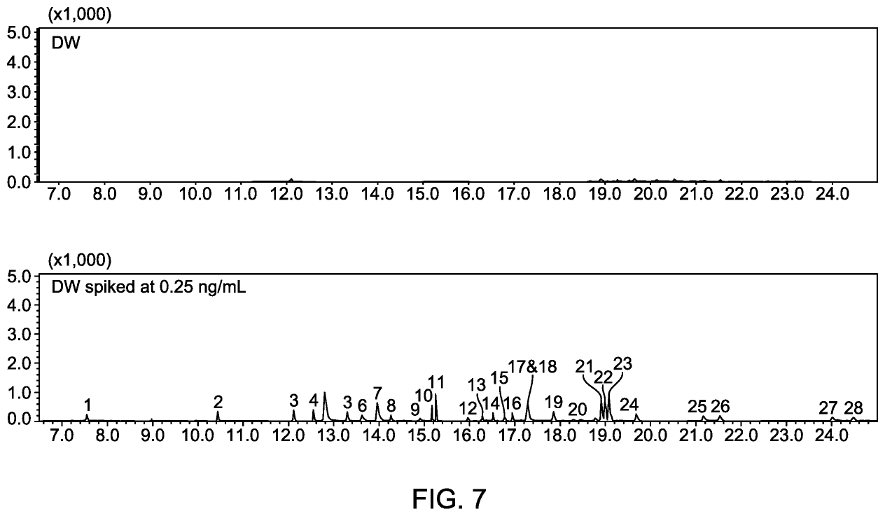

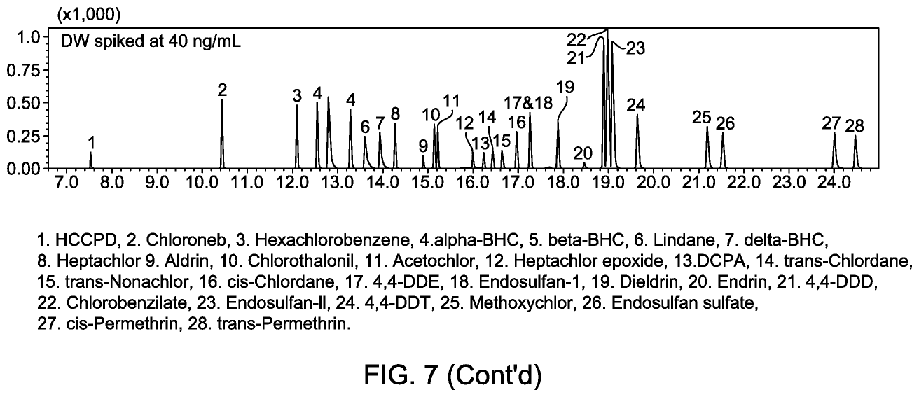

FIG. 7 illustrates GC-MS/MS chromatograms of DW sample and spiked DW samples. The peaks of the OCP were well separated at both spiked levels (0.25 ng/mL and 40 ng/mL). The peak area of OCP increased significantly when the spiked concentration was increased from 0.25 ng/mL to 40 ng/mL, which shows MILLE-GC-MS/MS method's applicability for a wide range of concentrations.

### Example 12: MILLE of OCP from OCP Standard Solution

A standard solution including a mixture of 29 OCP at 500 mg/L (RESTEK, EPA method 525.3 OCP) was used as a sample. A working standard solution of OCP with concentrations of 1 mg/L was prepared in acetone. 1 mL of the sample (2 ng/mL) was packed inside the porous membrane bag from the open end, and this end was then heat-sealed. The sample-packed porous membrane bag was placed in the 40 mL screw-capped glass vial, and 2 mL of the Hex solvent was added. The vial was closed tightly with a Teflon cap and agitated by shaking at 130 rpm for 60 min. The membrane bags were then removed from the glass vial, and Hex was evaporated to 50 μL and transferred for analysis by the MRM method.

### Example 13: MILLE of OCP from Various Water Sample Solution

1 mL of the samples (SWW, TW, and DW) were packed inside the porous membrane bag from the open end, and this end was then heat-sealed. The sample-packed porous membrane bags were placed in the 40 mL screw-capped glass vials, and 2 mL of the Hex solvent was added. The vials were closed tightly with the Teflon cap and agitated by shaking at 130 rpm for 60 min. The membrane bags were then removed from the glass vial, and Hex was evaporated to 50 μL and transferred for analysis.

A membrane-based inverted liquid-liquid extraction (MILLE) was developed and employed for the extraction of OCPs from drinking water samples. The effect of various factors on the extraction performance of OCPs was investigated. This method requires a very small sample volume packed inside a porous membrane bag. The extraction is performed by immersing the sample containing the membrane bag into a suitable solvent, whose volume can be reduced to enhance the concentration of analytes. MILLE eliminates many pre- and post-extraction steps. Also, it does not require the treatment of complex liquid samples, such as filtration or dilution, before being subjected to liquid-liquid extraction. After extraction, phase separation does not require additional steps such as centrifugation and filtration. It is applicable for small or large-volume samples. MILLE can even handle a few microliters of complex samples. The method described herein showed acceptable intra-day and inter-day precision in terms of percentage relative standard deviation (<15%). LODs were in the range of 0.03-0.08 ng/mL. This method was applied for the extraction of OCPs in SWW, DW, and TW samples, and the relative recoveries were found in the range of 81-120%. This method requires a small volume sample and has demonstrated the applicability in routine analysis.

Numerous modifications and variations of the present disclosure are possible in light of the above teachings. It is therefore to be understood that within the scope of the appended claims, the invention may be practiced otherwise than as specifically described herein.

1: A membrane-based inverted liquid-liquid extraction method for extracting an analyte from an unsupported aqueous liquid sample, the method comprising:
sealing the unsupported aqueous liquid sample in a porous membrane bag;
immersing the porous membrane bag in an organic solvent; and
extracting the analyte from the unsupported aqueous liquid sample to produce an extract within the organic solvent;
wherein the unsupported aqueous liquid sample is immiscible with the organic solvent; and
wherein the porous membrane bag does not contain a solid sorbent.

2: The method of claim 1, wherein the analyte is an organochlorine pesticide (OCP).

3: The method of claim 2, wherein the analyte is at least one organochlorine pesticide selected from the group consisting of hexachlorocyclopentadiene (HCCPD), chloroneb, hexachlorobenzene, alpha-benzenehexachloride (alpha-BHC), beta-benzenehexachloride (beta-BHC), gamma-benzenehexachloride (Lindane), delta-benzenehexachloride (delta-BHC), heptachlor, aldrin, chlorothalonil, acetochlor, heptachlorepoxide, dimethyl tetrachloroterephthalate (DCPA), trans-chlordane, trans-nonachlor, cis-chlordane, dichlorodiphenyldichloroethylene (4,4′-DDE), Endosulfan-I, Dieldrin, Endrin, dichlorodiphenyldichloroethane (4,4′-DDD), chlorobenzilate, Endosulfan-II, dichlorodiphenyltrichloroethane (4,4′-DDT), methoxychlor, Endosulfan sulfate, cis-permethrin, and trans-permethrin.

4: The method of claim 1, wherein the analyte is detected and/or quantified in a range of 0.25 to 80 nanograms (ng) per milliliter (mL) unsupported aqueous liquid sample.

5: The method of claim 1, wherein the analyte is present in the unsupported aqueous liquid sample at a concentration of 0.25 to 40 ng/mL.

6: The method of claim 1, wherein the porous membrane bag comprises a porous membrane having an average pore size of 0.05 to 1 micrometer (μm) and an average wall thickness of 10 to 500 μm.

7: The method of claim 6, wherein the porous membrane of the porous membrane bag has an average pore size of about 0.2 μm and an average wall thickness of about 157 μm.

8: The method of claim 1, wherein the porous membrane bag comprises at least one polymer selected from the group consisting of polypropylene, polyethylene, nylon, polyvinylidene fluoride, and polyethersulfone.

9: The method of claim 8, wherein the porous membrane bag consists of polypropylene.

10: The method of claim 1, wherein the organic solvent is at least one selected from the group consisting of acetone, benzene, cyclohexane, n-hexane, toluene, iso-octane, heptane, dichloromethane, and decane.

11: The method of claim 10, wherein the organic solvent is n-hexane.

12: The method of claim 1, wherein the unsupported aqueous liquid sample sealed in the porous membrane bag further comprises one or more acids and/or bases selected from the group consisting of nitric acid, sulfuric acid, phosphoric acid, hydrochloric acid, lithium hydroxide, potassium hydroxide, sodium hydroxide, and tetramethyl ammonium hydroxide.

13: The method of claim 1, wherein the unsupported aqueous liquid sample has a pH of 2 to 10.

14: The method of claim 1, wherein the unsupported aqueous liquid sample sealed in the porous membrane bag further comprises one or more inorganic salts selected from the group consisting of sodium chloride, sodium sulfate, sodium citrate, potassium chloride, potassium sulfate, and potassium citrate.

15: The method of claim 14, wherein the one or more inorganic salts are present in the unsupported aqueous liquid sample sealed in the porous membrane bag at a concentration of 0.1 to 1.5 moles per liter (mol/L).

16: The method of claim 1, wherein 0.02 to 0.2 mL of the unsupported aqueous liquid sample is present per square centimeters (cm²) exterior surface area of the porous membrane bag.

17: The method of claim 1, wherein the extracting is performed for 5 to 180 minutes.

18: The method of claim 1, having a limit of detection (LOD) in a range of 0.03 to 0.08 ng of the analyte per mL unsupported aqueous liquid sample.

19: The method of claim 1, having an analyte recovery of at least 90%.

20: The method of claim 1, further comprising:
removing the porous membrane bag from the organic solvent, and drying the organic solvent containing the extract to produce a concentrated extract solution; and
directly injecting the concentrated extract solution into a mass spectrometer or a chromatography column, wherein no filtering step and/or centrifuging step is used on the organic solvent, extract, or concentrated extract solution.
